(19) United States
(12) Patent Application Publication
(10) Pub. No.: US 2005/0165394 A1
(43) Pub. Date: Jul. 28, 2005

(54) Bone fixation system and method

(76) Inventors: Joseph Boyce, Lincoln, MA (US); Joseph Ting, Acton, MA (US); Thomas Fusco, Tewksbury, MA (US)

Correspondence Address: IANDIORIO & TESKA;INTELLECTUAL PROPERTY LAW ATTORNEYS, 260 BEAR HILL ROAD, WALTHAM, MA, 02451-1018, US

(21) Appl. No.: 11/038,802

(22) Filed: Jan. 20, 2005

Related U.S. Application Data
(60) Provisional application No. 60/538,826, filed on Jan. 23, 2004.

(51) Int. Cl. A61F 4/00

(52) U.S. Cl. 606/54

(57) ABSTRACT

A method of and system for attaching an orthopedic member to bone. The orthopedic member is positioned with respect to a bone segment. A plurality of pins are then driven through the orthopedic member and into the bone segment to secure the orthopedic member to the bone segment.

### RELATED APPLICATIONS

This application claims priority of Provisional Application Ser. No. 60/538,826 filed Jan. 23, 2004.

### FIELD OF THE INVENTION

This invention relates to a new method of attaching an orthopedic member such as a fixation plate to a bone segment.

### BACKGROUND OF THE INVENTION

One current method of bone fixation typically involves the use of a stainless steel or titanium fixation plate with five or six predrilled holes therethrough. The fixation plate is placed on a bone segment spanning a fracture. Holes are then drilled into the bone corresponding to the holes in the fixation plate. Screws are then driven through the fixation plate and into the bone segment.

Several problems exist with this method. First, there is a risk that the act of drilling the bone and/or installing the screws can cause damage to adjacent nerves or soft tissue. Second, the system is ineffective for small bones or comminuted fracture repair.

Also, load transfer and load distribution is often not optimized. Moreover, there can be a stiffness mismatch between the fixation plate and the bone which can lead to stress shielding. Sometimes, the screws can come loose. Placement of the screws is also severely limited because of the location and size of the pre-drilled holes in the fixation plate. There is also limited conformability of the fixation plate with respect to the bone resulting in stress concentrations. In general, current orthopedic fixation devices and systems are not very versatile.

### SUMMARY OF THE INVENTION

It is therefore an object of this invention to provide a new method of attaching an orthopedic member such as a fixation plate to bone.

It is a further object of this invention to provide such a method for attaching other types of orthopedic members to bone.

It is a further object of this invention to provide such a method which is more efficient and versatile.

It is a further object of this invention to provide such a method which does not require drilling in the bone.

It is a further object of this invention to provide such a method which lowers the possibility of causing damage to adjacent nerves or soft tissue.

It is a further object of this invention to provide such a method in which load transfer and load distribution is optimized.

It is a further object of this invention to provide such a method in which the fasteners used to secure the orthopedic member to the bone segment do not come loose.

It is a further object of this invention to provide such a method which provides a better stiffness match between the orthopedic member and the bone.

It is a further object of this invention to provide such a method which gives a surgeon options regarding placement of the fasteners.

It is a further object of this invention to provide an orthopedic member with better conformability lowering stress concentrations.

It is a further object of this invention to provide a system for attaching an orthopedic member to bone.

It is a further object of this invention to provide a system for the repair of small bones and bone fragments.

The subject invention results from the realization that instead of using screws and instead of pre-drilling the bone, a better and more versatile method of attaching an orthopedic member such as a fixation plate to a bone segment includes driving a plurality of pins through the orthopedic member and into the bone segment typically using ultrasonic energy.

The subject invention, however, in other embodiments, need not achieve all these objectives and the claims hereof should not be limited to structures or methods capable of achieving these objectives.

This subject invention features a method of attaching an orthopedic member to bone. The orthopedic member is positioned with respect to a bone segment and a plurality of pins are driven through the orthopedic member and into the bone segment to secure the orthopedic member to the bone segment. Typically, the bone segment need not be pre-drilled to accept the pins and the pins, when driven into the bone segment, do not cut internal threads therein.

In one example, the orthopedic member is a bone fixation plate positioned to span a defect such as a fracture in the bone segment. An array of pins are driven through the bone fixation plate and into the bone segment. Another orthopedic member is a spine fixation plate positioned to span at least two vertebrae. Still another example of an orthopedic member is a joint cup positioned in a bone socket. A further example of an orthopedic member is a bone staple. The method of the subject invention can also be used to position soft tissue between the orthopedic member and the bone and to drive the pins through both the orthopedic member and the soft tissue and into the bone to secure the soft tissue to the bone.

In one embodiment, the pins are first disposed in a body placed on the orthopedic member before driving the pins through the orthopedic member and into the bone. In one example, the body is made of a compactible material such as polymeric foam, ceramic foam, and metallic foam. In another example, the body is made of a more rigid material such as elastomers, metal, or plastic with preformed holes for receiving and supporting the plurality of pins.

A typical orthopedic member is made of metal, plastic, ceramic, or composite materials. In some embodiments, the orthopedic member has preformed holes which receive the plurality of pins. The pins can be made of silicon carbide, titanium, ceramic, stainless steel, hydroxyapatite, calcium phosphate, aluminum oxide, nitinol, carbon reinforced thermoplastics, or thermosets.

In one example, the pins are driven at least partially into the orthopedic member before the plate is positioned on the bone segment. Typically, the pins are then driven flush with the orthopedic member. The pins can be disposed perpendicularly through the orthopedic member or at an angle.

The pins can have many different configurations: surface knurling, bend-over heads, formed heads, thick and thin regions, pointed tips, angled shafts, pins made of porous material, and pins with drug dispensing capabilities.

Typically, driving the pins include using an ultrasonic horn to vibrate said pins. Pressure is usually applied to the pins by bearing down on the ultrasonic horn. Other examples include driving the pins using pneumatic or hydraulic devices. Typically, there are at least 100 pins every square inch of the orthopedic member.

A system for attaching an orthopedic member to a bone in accordance with the subject invention features at least one orthopedic member to be attached to a bone segment, a plurality of pins, and a driver for driving the pins through the orthopedic member and into the bone segment to secure the orthopedic member to the bone segment. In one preferred embodiment, an orthopedic member is positioned with respect to a bone segment and pins are ultrasonically driven through the orthopedic member and into the bone segment without drilling holes in the bone segment to secure the orthopedic member to the bone segment. Thus, one preferred system for attaching an orthopedic member to a bone in accordance with the subject invention features at least one orthopedic member to be attached to a bone segment, a plurality of pins, and an ultrasonic driver for driving the pins through the orthopedic member and into the bone segment to secure the orthopedic member to the bone segment.

### BRIEF DESCRIPTION OF THE DRAWINGS

Other objects, features and advantages will occur to those skilled in the art from the following description of a preferred embodiment and the accompanying drawings, in which:

FIG. 1 is schematic three-dimensional top view showing a typical prior art fixation plate attached to a bone segment with screws;

FIG. 2 is a schematic side cross-sectional view showing the fixation plate of FIG. 1 attached to a bone segment with screws;

FIG. 3 is a schematic three-dimensional front view of a typical prior art screw used to attach a fixation plate to a bone segment;

FIG. 4 is a three-dimensional schematic view of an ultrasonic driver useful in accordance with the method and system of the subject invention;

FIG. 5 is a schematic three-dimensional view of one example of a fixation plate in accordance with the subject invention;

FIG. 6 is a schematic front view showing a number of pins useful in accordance with the method and system of the subject invention;

FIG. 7 is a three-dimensional schematic view of the fixation plate shown in FIG. 5 and the pins shown in FIG. 6 ready to be attached to a bone segment in accordance with the subject invention;

FIG. 8 is a schematic three-dimensional view of another embodiment of a fixation plate in accordance with the subject invention;

FIG. 9 is a schematic three-dimensional view showing the fixation plate of FIG. 8 being secured to a bone segment using ultrasonic energy in accordance with the subject invention;

FIG. 10 is a schematic side cross-sectional view showing another embodiment of the subject invention wherein a compressible body is used to support the plurality of pins before they are driven through the fixation plate and into the bone segment in accordance with the subject invention;

FIG. 11 is a schematic cross-sectional side view showing use of an ultrasonic horn to drive the pins shown in FIG. 10 through the fixation plate and into the bone segment in accordance with the subject invention;

FIG. 12 is a schematic three-dimensional view showing another embodiment of a body for supporting the plurality of pins as they are driven through the fixation plate and into a bone segment in accordance with the subject invention;

FIG. 13 is a schematic three-dimensional view showing a plurality of pins pre-inserted into a plastic or composite fixation plate in accordance with the subject invention;

FIG. 14 is a schematic side cross-sectional view showing the use of pins to secure a joint cup to a bone socket in accordance with the subject invention;

FIG. 15 is a schematic three-dimensional view showing the use of pins in accordance with the subject invention in connection with a surgical staple;

FIG. 16 is a schematic top view showing the use of a plurality of pins to secure a spine fixation plate to vertebrae in accordance with the subject invention;

FIG. 17 is a schematic side cross-sectional view showing the use of pins in accordance with the subject invention used to secure a ligament to a bone segment;

FIG. 18 is a schematic cross-sectional side view showing how the pins can be angled or perpendicular to the orthopedic member in accordance with the subject invention; and

FIG. 19 is a schematic view showing a number of different pin configurations in accordance with the subject invention.

### DISCLOSURE OF THE PREFERRED EMBODIMENT

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

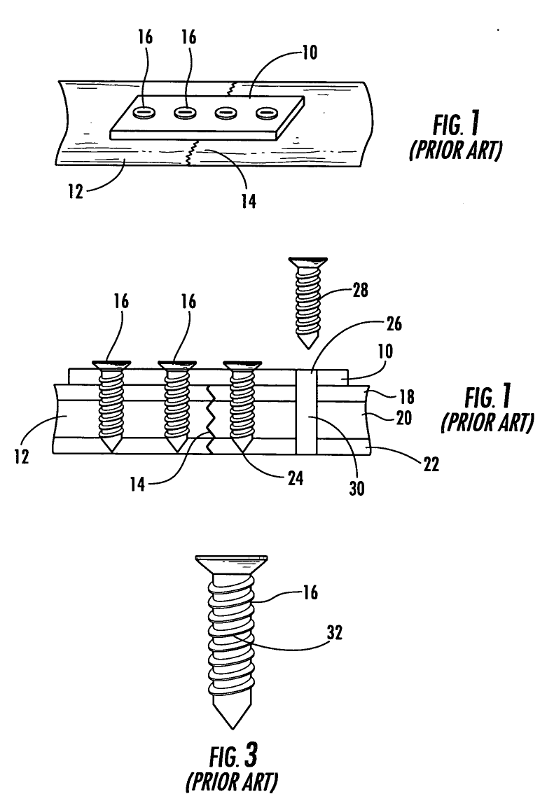

FIG. 1 shows typical prior art fixation plate 10 attached to bone segment 12 spanning fracture 14 using screws 16. Sometimes, screws 16, FIG. 2 extend through near cortice 18, through cancellous region 20, and into the cortex on the opposite side possibly resulting in adjacent nerve or tissue damage especially if the tip of a screw as shown at 24 breaks through the cortex on the opposite side 22. A typical plate 10 includes predrilled holes such as hole 26 and drill bit 28 is used to drill a corresponding hole 30 in bone 12. Screw 16, FIG. 3 is then driven through hole 26 in plate 10 and into hole 30 in bone 12 cutting internal threads in bone 12 when installed by the application of an applied rotational force translating into a resulting force along the longitudinal axis of screw 16 by virtue of threads 32.

Other problems with this current technique include the possibility that screws 16, FIGS. 1 and 2 can come loose. Load transfer and load distribution is also often not optimized. Moreover, there can be a stiffness mismatch between fixation plate 10 and bone segment 12. This can lead to a phenomena known as stress shielding in which the surrounding bone can become weak. Placement of the screws 16 is also limited because of the location of the pre-drilled holes in fixation plate 10. There is also limited conformability of fixation plate 10 with respect to bone segment 12 resulting in stress concentrations.

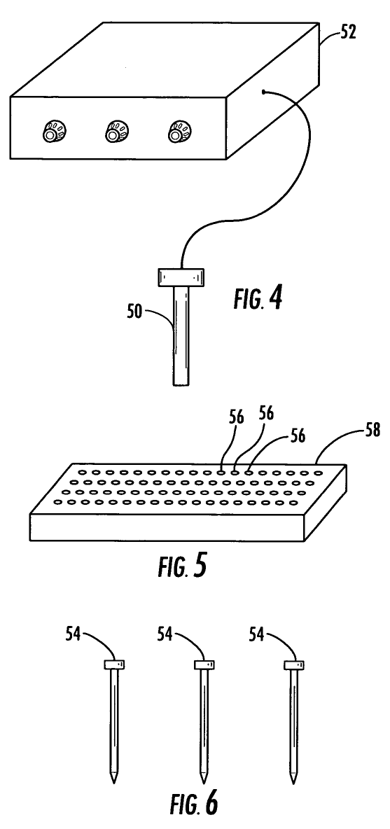

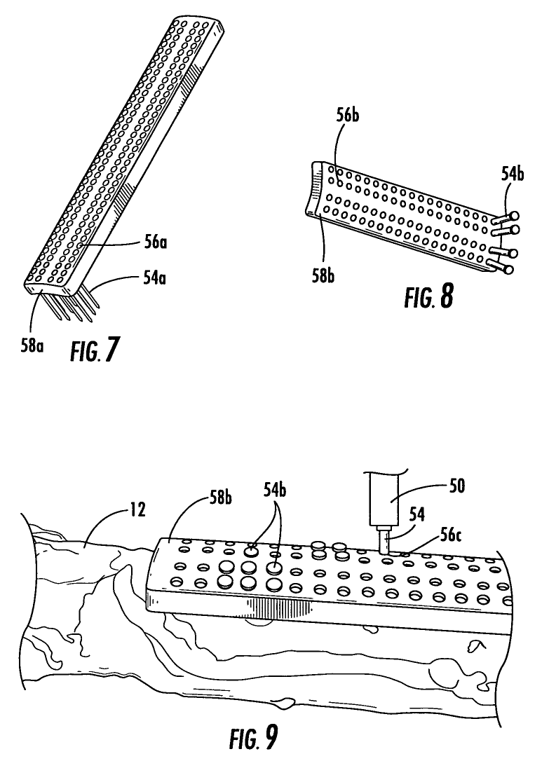

In one embodiment of the subject invention, in contrast, a driver such as ultrasonic horn 50, FIG. 4 made of titanium or hardened steel and powered by source 52 is used to drive a large number of pins 54, FIG. 6 through preformed holes 56 in fixation plate 58, FIG. 5 and into a bone segment. The pins are securely retained in the bone due to the inherent compression properties of the bone which generates local compressive forces around each pin after insertion. A suitable ultrasonic horn is available from Aztex, Inc. (Waltham, MA) as the UAZ™-2000 pin insertion device driven at 20-40 kHz, 100% power, 85% amplitude. Typically, four or more pins are driven through the plate and into the bone segment in unison. In one example, shown in FIG. 7, 77 mm long, 10 mm wide, 3 mm thick curved plate 58 was made of Teflon and included an array of four rows of 36 predrilled holes 56a. There are 144 silicon carbide pins 54a available to the surgeon 0.5 mm in length and 0.0056-0.010 inches in diameter spaced 1 mm apart. Not all the pins may be used in certain surgical procedures, however. In this way, the surgeon can decide where best to place pins 54a for secure attachment of plate 58a to a bone segment. In another example, 10 mm wide, 73 mm long, 3 mm thick plate 58b, FIG. 8 included an array of four rows of 19 holes 56b and there were 76 pins 54b. Plate 59b, FIG. 9 is shown secured to bone segment 12 in an experiment with several pins 54b are already installed. Pin 54c is shown assembled with plate 58b positioned in hole 56c and being driven through plate 58b and into bone segment 12 by ultrasonic horn 50. Typically, there were 100-150 pins which could be used every square inch of fixation plate 58b.

By not requiring that holes be pre-drilled in bone 12, and by using a large plurality of small diameter pins in contrast to only 4-8 large diameter screws and by using ultrasonic energy to drive the small diameter pins through the fixation plate and into the bone, several advantages are realized in accordance with the subject invention. First, there is a less chance of damage to adjacent nerves or soft tissue. Pre-drilling holes in the bone is not required. The pins are also easier to install. The pins also do not loosen as readily as screws. Pin placement is not as limited as in the prior art since there are numerous pre-drilled holes in plate 58b. Now the surgeon can select the desired pin locations from among the many holes in plate 58b. In general, the retention strength afforded by the use of pins in accordance with the subject invention is greater than or at least equal to the case where screws are used.

Moreover, small (e.g., 20 mil) diameter pins can be used when large screws cannot such as in a comminuted fracture or in connection with a small diameter bone. The pins do not cut internal threads in the bone segment and instead are driven in by the combination of vibration and pressure and/or heat. Care should be taken to minimize burning of the bone immediately adjacent a pin, however.

In simulation testing, an array of 16 pins had an average maximum pullout load of 9.7 kgf for flat plates and above 14.2 kgf for curved plates.

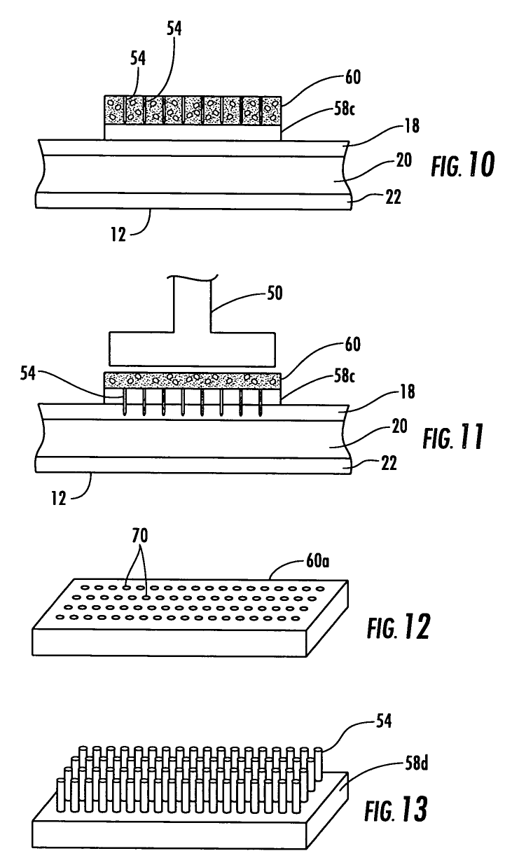

In another example, pins 54, FIG. 10 are first inserted in compactible (e.g., foam) body 60 which is then placed on fixation plate 58c itself positioned on bone segment 12. In this example, fixation plate 58c is made of plastic and does not have any preformed holes. Foam body 60 supports the pins as they are driven through fixation plate 58c, FIG. 11 and into near cortice region 18 of bone 12 by ultrasonic horn 50. As ultrasonic horn 50 bears down on pins 54 and vibrates them through plate 58c and into bone 12, foam body 60 collapses. Foam body 60 can then be removed and, if any pin heads are not flush with plate 58c, they are driven flush by horn 50. Materials for foam body 60 may include degradable surgical foam, polymeric foam, ceramic foam, and even metallic foam. Foam body 60 may also include layers of different density foam.

Pins 54c can be made of silicon carbide, titanium, ceramic materials, cobalt chrome, stainless steel, hydroxyapatite, calcium phosphate, aluminum oxide, nitinol, carbon reinforced, thermoplastics, and thermosets.

Plate 58c can be made of plastic or composite materials. In one experimental example, a PTFE plate was used with silicon carbide pins to provide better flexural and torsional stiffness properties which are more like those of bone.

Instead of a highly compactible material, such as foam, body 60a, FIG. 12 can be a more rigid material made of, for example, an elastomer, metal, or plastic. Body 60a has preformed holes 70 which receive the pins to support them as the ultrasonic horn is used to drive the pins through the fixation plate and into the bone. The fixation plate may include preformed holes or not depending on the materials used for the plate and the pins.

In still another example, pins 54 are partially driven into plastic or composite fixation plate 58d, FIG. 13 using ultrasonic energy and/or pressure or by using a hydraulic or pneumatic driver and then fixation plate 58d is positioned on a bone segment. Thus, in this example, fixation plate 58d itself is used to support pins 54 as they are driven further through fixation plate 58d and into a bone segment by a suitable driver (e.g., ultrasonic, hydraulic, or pneumatic). Experiments with polyethylene, PEEK, PTFE, and Teflon plates showed that the pins could be supported in a foam body and inserted first into the plastic plate relying on localized melting of the plastic material of the plate around the pin insertion sites when ultrasonic energy is applied to the pin heads. This plate containing the projecting pins was then positioned on the bone segment to span a bone defect such as a fracture and a second application of ultrasonic energy to the heads of the pin array was used to insert the pins into the bone flush with the top surface of the plate.

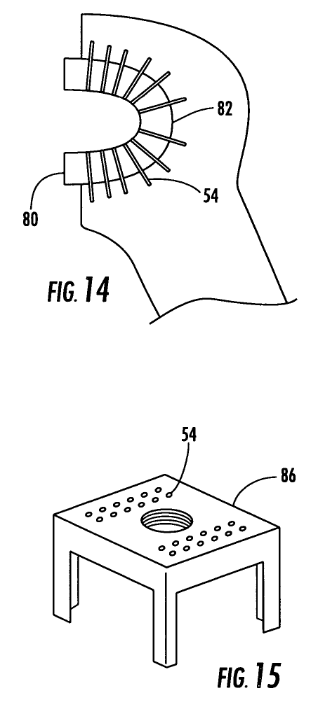

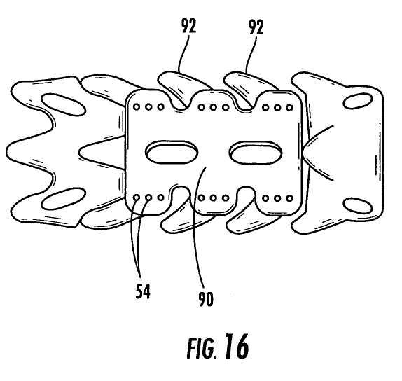

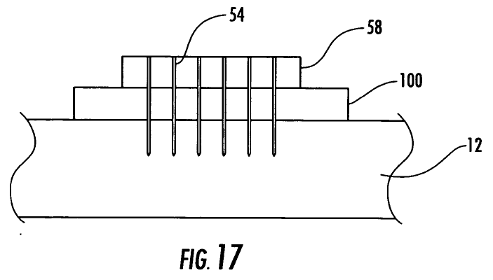

Other orthopedic members which can be secured to bone material in accordance with the subject invention include joint cup 80, FIG. 14 secured to bone socket 82 by pins 54. An array of small diameter pins 54 are inserted through cup 80 to improve load distribution and eliminate the need for pre-drilling of holes in bone socket 82. Also, orthopedic bone staple 86, FIG. 15 can be secured in place using pins 54. Spine fixation plate 90, FIG. 16 spanning vertebrae 92 can also be fixed in place in accordance with the subject invention. In cases where the spinal discs have deteriorated, lumbar vertebrae 92 are fixed using plate 90 and pins 54. Installation of pins 54 do not require pre-drilling and can be used to obtain adequate mechanical strength with reduced depth of penetration relative to screws which require drilling into the pedicles of the vertebrae with the risk of break through and damage to the patient's spinal cord.

Also, pins 54 can be driven through both orthopedic plate 58, FIG. 17 and soft tissue such as ligament 100 and into bone 12 in accordance with the subject invention to secure ligament 100 to bone 12. Tendons or other soft tissue structures can also be secured in this manner.

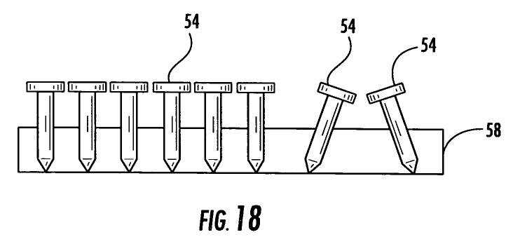

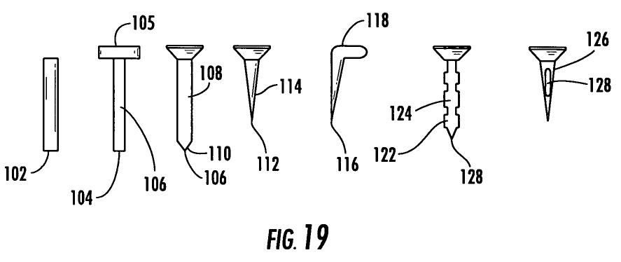

Thus, the subject invention yields a highly versatile fixation method including the ability to position pins 54, FIG. 18 perpendicular in fixation plate 58 or at any angle as shown. Indeed, various types of pins can be used including straight headless pin 102, FIG. 19; pin 104 with a formed head 105 and with surface knurling 106 on the shaft thereof; headed pin 106 with straight shaft 108 and pointed tip 110; headed pin 112 with angled shaft 114; pin 116 with bent over head 118; pin 120 with thick 122 and thin 124 regions to promote bone growth attachment to the pin; and pin 126 made of biodegradable material and including drug delivery cavity 128. The pins can also be coated with various materials and/or can be made of highly porous materials to promote bone growth.

Concept feasibility has been demonstrated using both model cancellous bone mazenal and cadaver bones. Arrays of steel pins driven straight into blocks of model cancellous bone had the same or higher pullout strengths as 3.5 mm bone screws which had the same cross-sectional area as the pins. Arrays of slightly angled pins showed significantly higher pullout loads. Identical sized bone fixation plates, one designed for standard 3.5 mm bone screws and one designed for an array of 0.020 in diameter pins were used to repair canine cadaver radii. The bones were cut in half using an oscillating bone saw and then repaired with plates fixed with screws and plates. The two systems showed equivalent performance as the bones were tested for four point bend response.

In this way, the use of pins in accordance with the subject invention can provide superior bone fixation compared to current techniques. Screw fixation is inefficient from both a structural and biological standpoint. In the case of prior art fracture plates, screws must be placed along the entire length of a plate in order to provide adequate mechanical properties against bending, shear, and pull-out loads. And, application of a plate to a fractured femur requires surgeons to make an incision to expose the entire femur.

The use of an array of pins in accordance with the subject invention provides a much more efficient fixation method and spreads the load and provides for superior mechanical performance for the same fixation area as screws. The pins are capable of penetrating cortical bone and arrays of pins provide a holding power at least comparable to conventional screws. Pin removal, if desired, can be addressed through mechanical design, for example, by adding heads to the pins. The pins are typically inserted using ultrasonic equipment which creates local heating. Although excessive heat can lead to protein breakdown, conventional drilling has shown that some degree of damage is biologically acceptable.

The result is a new method of attaching an orthopedic member such as a fixation plate and other types of orthopedic members to bone. The method is more efficient because drilling in the bone is not required. The possibility of causing damage to adjacent nerves or soft tissue is reduced. Load transfer and load distribution are optimized. The pins used to secure the orthopedic member to the bone segment do not typically come loose. There is also now the ability to provide a better stiffness match between the orthopedic member and the bone. Surgeons now have options regarding placement of the fasteners. Moreover, the orthopedic member can be designed with better conformability lowering stress concentrations.

The system and method of the subject invention features attaching an orthopedic member such as a fixation plate to a bone segment by driving a plurality of pins through the orthopedic member and into the bone segment typically using ultrasonic energy. The materials used for and the configuration of the pins and the orthopedic member can vary widely rendering the subject invention highly versatile.

Although specific features of the invention are shown in some drawings and not in others, however, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

1. A method of attaching an orthopedic member to bone, the method comprising:
positioning the orthopedic member with respect to a bone segment;
assembling a plurality of pins with the orthopedic member; and
driving the pins through the orthopedic member and into the bone segment to secure the orthopedic member to the bone segment.

2. The method of claim 1 in which the bone segment is not pre-drilled to accept the pins.

3. The method of claim 1 in which the pins, when driven into the bone segment, do not cut internal threads therein.

4. The method of claim 1 in which the orthopedic member is a bone fixation plate positioned to span a defect in the bone segment.

5. The method of claim 4 in which there are an array of pins driven through the bone fixation plate and into the bone segment.

6. The method of claim 1 in which the orthopedic member is a spine fixation plate positioned to span at least two vertebrae.

7. The method of claim 1 in which the orthopedic member is a joint cup positioned in a bone socket.

8. The method of claim 1 in which the orthopedic member is a staple.

9. The method of claim 1 further including the steps of positioning soft tissue between the orthopedic member and the bone and driving the pins through both the orthopedic member and the soft tissue and into the bone to secure the soft tissue to the bone.

10. The method of claim 1 further including the step of disposing the pins in a body placed on the orthopedic member before driving the pins through the orthopedic member and into the bone.

11. The method of claim 10 in which the body is made of a compactible material.

12. The method of claim 11 in which the compactible body is made of a material selected from the class consisting of polymeric foam, ceramic foam, and metallic foam.

13. The method of claim 10 in which the body is made of a more rigid material and includes preformed holes for receiving and supporting the plurality of pins.

14. The method of claim 13 in which the more rigid body is made of a material selected from the class consisting of elastomers, metal, or plastic.

15. The method of claim 1 in which the orthopedic member is made of material selected from the class consisting of metal, plastic, ceramic, and composite materials.

16. The method of claim 1 in which the orthopedic member includes preformed holes which receive the plurality of pins.

17. The method of claim 1 in which the pins are made from material selected from the class consisting of silicon carbide, titanium, ceramic, stainless steel, cobalt chrome, hydroxyapatite, calcium phosphate, aluminum oxide, silicon carbide, nitinol, carbon reinforced thermoplastics, and thermosets.

18. The method of claim 1 in which the pins are driven at least partially into the orthopedic member before the plate is positioned on the bone segment.

19. The method of claim 1 in which the pins are driven flush with the orthopedic member.

20. The method of claim 1 in which the pins are disposed perpendicularly through the orthopedic member.

21. The method of claim 1 in which the pins are disposed at an angle through the orthopedic member.

22. The method of claim 1 in which the pins have configurations from the class consisting of surface knurling, bent-over heads, formed heads, thick and thin regions, pointed tips, angled shafts, pins made of porous material, and pins with drug dispensing capabilities.

23. The method of claim 1 in which driving the pins include using an ultrasonic horn to vibrate said pins.

24. The method of claim 23 in which pressure is applied to the pins by bearing down on the ultrasonic horn.

25. The method of claim 1 in which driving the pins includes pneumatically or hydraulically driving the pins.

26. The method of claim 1 in which there are at least 100 pins every square inch of the orthopedic member.

27. A system for attaching an orthopedic member to a bone, the system comprising:
at least one orthopedic member to be attached to a bone segment;
a plurality of pins; and
a driver for driving the pins through the orthopedic member and into the bone segment to secure the orthopedic member to the bone segment.

28. The system of claim 27 in which the orthopedic member is a bone fixation plate.

29. The system of claim 28 in which there are an array of preformed holes in the fixation plate for receiving the pins.

30. The system of claim 27 in which the orthopedic member is a spine fixation plate.

31. The system of claim 27 in which the orthopedic member is a joint cup.

32. The system of claim 27 in which the orthopedic member is a staple.

33. The system of claim 27 further including a body to be placed on the orthopedic member for supporting the pins as they are driven through the orthopedic member and into the bone.

34. The system of claim 33 in which the body is made of a compactible material.

35. The system of claim 34 in which the compactible body is made of a material selected from the class consisting of polymeric foam, ceramic foam, and metallic foam.

36. The system of claim 33 in which the body is made of a more rigid material and includes preformed holes for receiving the plurality of pins.

37. The system of claim 36 in which the more rigid body is made of a material selected from the class consisting of elastomers, metal, or plastic.

38. The system of claim 27 in which the orthopedic member is made of material selected from the class consisting of metal, plastic, ceramic, or composite materials.

39. The system of claim 27 in which the pins are made from material selected from the class consisting of silicon carbide, titanium, ceramic, stainless steel, hydroxy apatite, calcium phosphate, aluminum oxide, silicon carbide, nitinol, carbon reinforced, thermoplastics, and thermosets.

40. The system of claim 27 in which the pins are driven at least partially into the orthopedic member.

41. The system of claim 40 in which the pins are driven perpendicularly with respect to the orthopedic member.

42. The system of claim 40 in which the pins are driven at an angle in the orthopedic member.

43. The system of claim 27 in which the pins have configurations from the class consisting of surface knurling, bend-over heads, formed heads, thick and thin regions, pointed tips, angled shafts, pins made of porous material, and pins with drug dispensing capabilities.

44. The system of claim 27 in which the driver includes an ultrasonic horn.

45. The system of claim 27 in which the driver includes a pneumatic or hydraulic device.

46. A method of attaching an orthopedic member to bone, the method comprising:
positioning the orthopedic member with respect to a bone segment;
assembling a plurality of pins with the orthopedic member; and
ultrasonically driving the pins through the orthopedic member and into the bone segment without drilling holes in the bone segment to secure the orthopedic member to the bone segment.

47. The method of claim 46 in which the orthopedic member includes an array of pre-formed holes for receiving the pins.

48. A system for attaching an orthopedic member to a bone, the system comprising:
at least one orthopedic member to be attached to a bone segment;
a plurality of pins; and
an ultrasonic driver for driving the pins through the orthopedic member and into the bone segment to secure the orthopedic member to the bone segment.

49. The system of claim 1 in which the orthopedic member includes an array of pre-formed holes for receiving the pins.
